(19) United States
(12) Patent Application Publication
(10) Pub. No.: US 2026/0194397 A1
(43) Pub. Date: Jul. 9, 2026

(54) SAFETY COVER FOR MONITORING ELECTROCHEMICAL BATTERIES, WITH TEMPERATURE SENSORS

(71) Applicant: COMMISSARIAT A L'ÉNERGIE ATOMIQUE ET AUX ENERGIES ALTERNATIVES, Paris (FR)

(72) Inventors: Aurélia DITTO, Grenoble Cedex 09 (FR); Céline BONNAUD, Grenoble Cedex 09 (FR); Clément WEICK, Grenoble Cedex 09 (FR); Julien DAUCHY, Grenoble Cedex 09 (FR)

(73) Assignee: COMMISSARIAT A L'ÉNERGIE ATOMIQUE ET AUX ENERGIES ALTERNATIVES, Paris (FR)

(21) Appl. No.: 19/438,865

(22) Filed: Jan. 2, 2026

(30) Foreign Application Priority Data
Jan. 7, 2025 (FR) FR2500137

(51) Int. Cl. G01K 3/00; G01K 1/02; G01K 1/024; G01N 33/00; G01R 31/12; H01M 10/42

(52) U.S. Cl. CPC G01K 3/005; G01K 1/024; G01K 1/026; G01N 33/0063; G01R 31/1218; H01M 10/4285

(57) ABSTRACT

A safety cover for monitoring electrochemical batteries, including a flexible membrane, able to at least partially surround at least one electrochemical battery to be monitored by defining a confined atmosphere, this flexible membrane having an inner face intended to face the electrochemical battery, and an outer face intended to face the outside; at least one temperature sensor disposed on the inner face of the flexible membrane; a control module connected to the temperature sensor and able to detect an event involving an increase in the temperature of the confined atmosphere at a rate exceeding a predetermined threshold; an alert interface able to emit a signal representative of an alert when the control module detects the event.

### TECHNICAL FIELD

The invention relates to the field of electrochemical batteries used in numerous fixed or mobile applications for the storage of electrical energy. These applications include, for example, electric vehicles, light mobility vehicles, stationary electrical energy accumulators, buffer storage for renewable energy solutions (photovoltaic panels, wind turbines, etc.), or any other fixed or mobile means of storing electrical energy.

The invention relates more specifically to means for monitoring these batteries, from a safety perspective.

The rapid growth in the number of devices, installations and vehicles equipped with electrochemical batteries across all power ranges has necessitated adaptations to address safety issues arising from the presence of batteries in a wide variety of locations (relating to mobility devices and portable objects) and the presence of large quantities of batteries and/or high-power batteries in user environments, in transport, or in places where these batteries are processed.

Indeed, electrochemical batteries present potential risks of corrosive and/or flammable electrolyte leakage, thermal runaway, fire, explosion, etc.

Electrochemical batteries include lithium or nickel batteries, lead-acid batteries, etc., or any other common type of electrochemical battery. The term “electrochemical battery” here encompasses individual cells, modules comprising several cells, batteries comprising several modules, battery packs comprising several such batteries, or any other battery sub-assembly.

### PRIOR ART

Currently known devices for monitoring electrochemical batteries are generally based on the battery's BMS (Battery Management System) devices, or on other electronic devices related to the batteries or battery groups used.

Known devices provide a satisfactory first level of safety when batteries are equipped with such devices. However, these devices may fail when the battery itself is faulty.

For certain applications, for certain batteries not equipped with these devices, and for situations where the battery's composition is unknown (recycling, transport, prevention, accident phases, etc.), known means do not provide a sufficient level of safety.

### DESCRIPTION OF THE INVENTION

The invention aims to improve safety measures for monitoring electrochemical batteries.

To this end, the invention provides a safety cover for monitoring electrochemical batteries, comprising:
a flexible membrane, able to at least partially surround at least one electrochemical battery to be monitored by defining a confined atmosphere, this flexible membrane having an inner face intended to face the electrochemical battery, and an outer face intended to face the outside;
at least one temperature sensor disposed on the inner face of the flexible membrane;
a control module connected to the temperature sensor and able to detect an event involving an increase in the temperature of the confined atmosphere at a rate exceeding a predetermined threshold;
an alert interface able to emit a signal representative of an alert when the control module detects said event.

According to another objective, the invention relates to a method for monitoring an electrochemical battery, with a safety cover as described above, comprising the following steps:
positioning the safety cover on the battery;
detecting, using the control module, an event involving an increase in the temperature of the confined atmosphere at a rate exceeding a predetermined threshold;
activating the alert interface.

The invention enables electrochemical batteries to be monitored with an increased level of safety, independently of the batteries themselves that are being monitored, the environmental conditions of these batteries, the identification of these batteries, and their history.

The invention adds a high level of safety to batteries that lack internal diagnostic capabilities, batteries with faulty monitoring capabilities, or batteries of which the composition is unknown.

The invention is particularly suitable for life phases outside the nominal discharge phases, for example recycling phases, accident prevention, securing batteries involved in an incident, securing vehicles or installations, and transporting batteries or vehicles, machines or installations.

However, the invention can be implemented in all phases of battery use, particularly during charging phases, which may require monitoring.

For example, the life phases relating to battery recycling will become increasingly widespread and will be subject to a growing number of processes, associated storage, and appropriate recycling plants, the size of which will increase. These recycling phases are regulated and even mandatory. In this example, large quantities of high-power batteries will be transported and stored for recycling processes, and these batteries may be of unknown composition, operation, and history. The invention makes it possible to secure this type of battery stock with uncertain properties by detecting and alerting to potential or actual danger.

In another example, the transport phases of batteries are complex phases requiring specific identification and measurements, as well as approvals or standardisation with regard to transporters. The invention enables any haulier to add a level of safety to the transport service, or even to obtain approval that would not be obtained without the safety cover, even for batteries of unknown nature and history.

According to another example, in the event of an incident (accident, fire, flood, etc.) involving a facility or vehicle containing batteries, potential damage to the batteries may trigger undesirable events at a later stage, such as thermal runaway and fire, electrolyte projection, degassing, etc. The invention makes it possible to secure an accident site by monitoring the batteries in order to protect emergency responders and avoid creating aggravating factors.

In another example, when transporting a damaged facility or a vehicle that has been damaged or involved in a collision, the transport phases of these potentially dangerous items can be made safe by the invention.

According to another example, the charging phases of a battery can be risky sequences, due to disruption of the surrounding environment, possible electrical faults, charger failure, etc. The invention also makes it possible to dynamically secure these charging phases.

The invention also complements battery monitoring with an alert for all hazards that may occur to a battery, such as: mechanical shocks, deep discharge, overcharging, external short circuits, and excessively high ambient temperatures.

The invention offers a practical and easy-to-implement solution that does not require technical training.

The invention also allows for great modularity in the implementation of alerts, which can be audible, visual, remote, transmitted over a network, etc.

The safety cover according to the invention may comprise the following additional features, individually or in combination:
said at least one temperature sensor comprises an array of temperature sensors arranged on the inner face of the flexible membrane and connected to the control module;
the array of temperature sensors consists of a surface matrix of temperature sensors, arranged on the surface of the inner face of the flexible membrane;
the array of temperature sensors has a surface density of sensors of substantially 1 sensor per 10 cm² on at least a portion of the inner face of the flexible membrane;
the alert interface is able to emit a signal representing an alert indicating the location of the sensor zone in which the event was detected;
the safety cover also includes at least one carbon oxide sensor located on the inner face of the flexible membrane, and the control module is connected to said at least one carbon oxide sensor and is able to detect an event involving an increase in the level of carbon oxide gas in the confined atmosphere at a rate exceeding a predetermined threshold, the alert interface being able to emit a signal representative of an alert when the control module detects said event;
the carbon oxide sensor is a carbon dioxide and carbon monoxide level sensor;
the flexible membrane is formed of a flexible material of which the melting point is higher than a predetermined temperature;
the flexible membrane has a weighted peripheral rim;
the alert interface comprises an audible alert device or a visual alert device;
the alert interface comprises a remote alert device;
the alert interface is able to emit a differentiated signal depending on the event detected by the control module;
the flexible membrane is made of a fabric of glass fibres, or silica, or silica aerogel;
the flexible membrane is coated with a polymer or mineral;
the safety cover also comprises an array of light intensity sensors arranged on the inner face of the flexible membrane, and the control module is able to also detect an event of an increase in light intensity at a speed greater than a predetermined threshold;
the array of light intensity sensors has a surface density of sensors of substantially 1 sensor per 50 cm² on at least a portion of the inner face of the flexible membrane;
the flexible membrane is opaque;
the safety cover also comprises at least one accelerometer arranged on the inner face of the flexible membrane, and the control module is able to also detect an impact event experienced at an acceleration greater than a predetermined threshold;
the safety cover further comprises at least one volatile organic compound sensor disposed on the inner face of the flexible membrane, and the control module is able to further detect an event involving the presence of a carbonated compound;
the safety cover further comprises at least one ambient temperature sensor arranged on the outer face of the flexible membrane, in contact with the ambient air, and the control module is able to further detect an event of an increase in the ambient temperature beyond a predetermined threshold;
the alert interface further comprises a cut-off module able to disconnect battery charging equipment and/or to trigger a battery inerting system, and/or to trigger a battery extinguishing system;
the alert interface is able to emit a signal representative of an alert indicating an alert level.

### PRESENTATION OF THE FIGURES

Other features and advantages of the invention will become apparent from the following non-limiting description, with reference to the appended drawings, in which:

FIG. 1 schematically illustrates a perspective view of a battery intended to be monitored;

FIG. 2 illustrates a safety cover placed over the battery;

FIG. 3 schematically illustrates the safety cover laid flat, from the side of its inner face;

FIG. 4 schematically illustrates the safety cover laid flat, from the side of its outer face;

FIG. 5 is a schematic cross-sectional view of the safety cover placed on the battery;

FIG. 6 is a diagram illustrating the method for monitoring batteries

FIG. 7 is a diagram illustrating variants of the method.

Elements that are similar and common to the various embodiments bear the same reference numerals referring to the figures.

### DETAILED DESCRIPTION

The invention relates to a safety cover 1 for monitoring electrochemical batteries. This safety cover 1 is intended to cover, directly or indirectly, at least part of a battery 2, or a group of batteries, that is to be monitored.

The safety cover 1 is intended for the exceptional or systematic monitoring of an electrochemical battery in order to monitor, in particular, any thermal runaway, fire, degassing, etc.

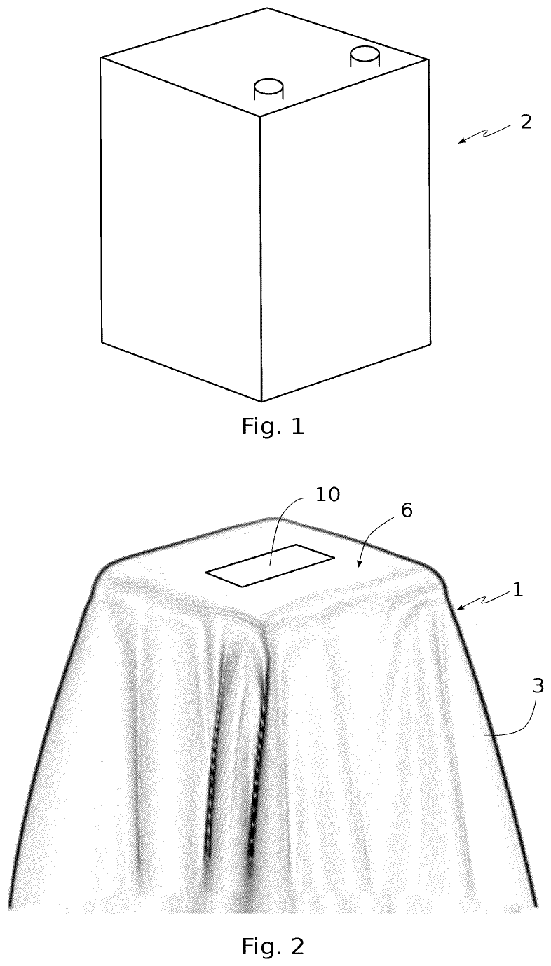

The safety cover 1 is placed directly on the battery 2 or on a sub-assembly containing the battery 2, or even on a vehicle containing the battery 2. FIG. 1 illustrates an example of an electrochemical battery 2 that is to be monitored.

For example, the battery 2 may be a lithium-ion battery being monitored for storage or transport prior to recycling, or to secure equipment or a vehicle involved in an accident at the scene of the accident, or during the transport of damaged components.

The safety cover 1 comprises a flexible membrane 3 that is able to at least partially surround the battery 2. FIG. 2 illustrates an example in which the battery 2 from FIG. 1 is placed on a surface, and the safety cover 1 is arranged so as to completely cover the battery 2.

The flexible membrane 3 is designed to cover the battery and is made of a suitable flexible material such as an industrial textile, woven or non-woven.

Preferably, the material of the flexible membrane 3 is resistant to high temperatures or is even fire-resistant. More specifically, the membrane 3 is preferably made of a flexible material of which the melting point is higher than a predetermined temperature. For example, this predetermined temperature may be approximately 800° C. when the flexible membrane 3 is made of a glass fibre fabric with a melting point of around 800° C.

The flexible membrane 3 has the following main features:
it is able to create a confined atmosphere 8 around the battery 2. Absolute gas tightness is not necessary; it is sufficient for the flexible membrane 3 to produce relative confinement, even with relative tightness such as that provided by a woven fabric;
it has sufficient mechanical strength to support the sensors 4, 7 described below;
it is sufficiently flexible to cover the battery. The safety cover 1 can advantageously cover different types and shapes of batteries, within a certain size range, in order to be adaptable.

The flexible membrane 3 can be made from any industrial textile having these features. It may advantageously be a silica aerogel, a glass fibre fabric or a silica fabric, which are fireproof and resistant to perforation. These fabrics may advantageously be coated with a polymer or mineral, for example polyurethane or vermiculite.

The flexible membrane 3 has an inner face 5 intended to face the battery 2 when the safety cover 1 is in place thereon, and an outer face 6 facing outwards and visible or accessible to the user.

The safety cover 1 may comprise on its inner face 5:
at least one temperature sensor 4, or even an array of a plurality of temperature sensors 4; and/or
at least one carbon oxide sensor 7, or even an array of a plurality of carbon oxide sensors 7.

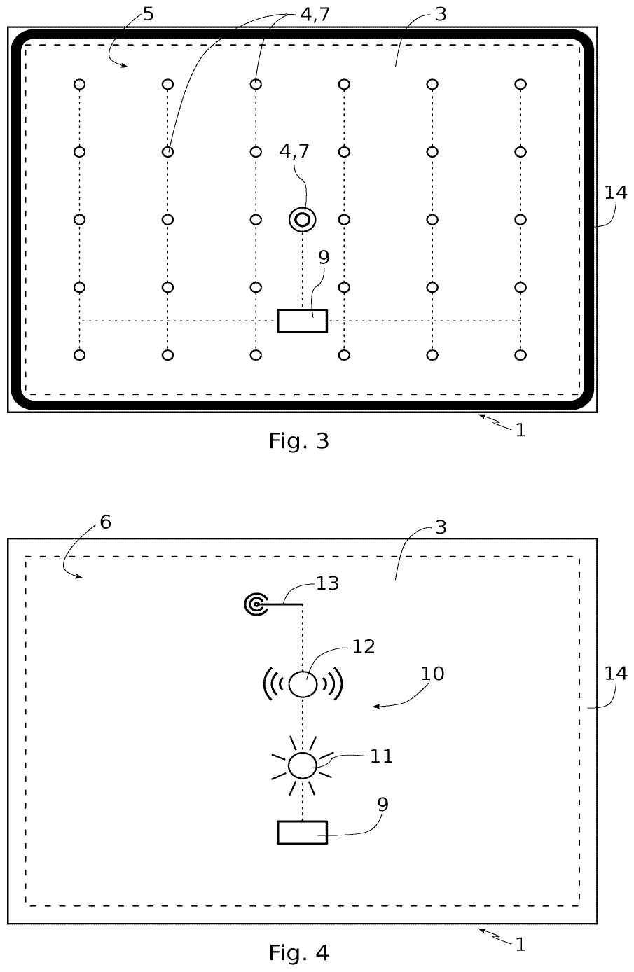

FIG. 3 is a plan view of a safety cover 1 deployed and laid flat, with the inner face 5 visible. The array of temperature sensors 4 and/or carbon oxide sensors 7 is shown schematically, along with its electrical connections indicated by dotted lines. These connections may be in the form of cables or other means of communication.

In this example, the array of sensors 4, 7 consists of a surface matrix of sensors 4, 7 arranged on the inner face 5 of the flexible membrane 3.

FIG. 3 illustrates the case where the safety cover 1 comprises an array of sensors 4, 7 which is advantageously centred on the inner face 5.

FIG. 3 also illustrates the case where the safety cover 1 has a single sensor 4, 7, which is advantageously located in a central position on the inner face 5.

These two cases can be combined.

The array of sensors 4, 7 is intended to cover at least part of the confined atmosphere 8 (the atmosphere confined by the flexible membrane 3). The finer the array, the more effective the monitoring, but the more sensors 4, 7 are required.

The array of sensors 4, 7 is defined as comprising at least one sensor 4, 7 (suitable, for example, in the case of a small safety cover 1 for monitoring a small battery 2). The array comprises a number of sensors 4, 7 adapted to the maximum battery dimensions for which the safety cover 1 is intended.

Advantageously, the array of sensors 4, 7 has a surface density of sensors of substantially 1 sensor per 10 cm² of flexible membrane 3, on at least a portion of the inner face 5 of the flexible membrane 3.

In the illustrative example of FIG. 3, the safety cover 1 comprises an array of thirty sensors 4, 7 on the inner face 5 of the flexible membrane 3. The array of sensors 4, 7 is shown here in the form of a matrix of sensors 4, 7 aligned in rows and columns. Alternatively, the sensors 4, 7 may be distributed according to any other appropriate geometric pattern, or even randomly distributed, for a particular battery shape, for example in a spiral, in concentric circles, etc.

Each of the sensors 4, 7 in the array is attached to the flexible membrane 3 by any known means, such as sewing, gluing, crimping, etc. The sensors 4, 7 may also be integrated into the fabric forming the flexible membrane 3 by incorporating microsensors into the weave.

The array of sensors 4, 7, in its surface arrangement, makes it possible to monitor the localized evolution of the temperature or carbon oxide level in the confined atmosphere 8, for the purpose of the treatment described below.

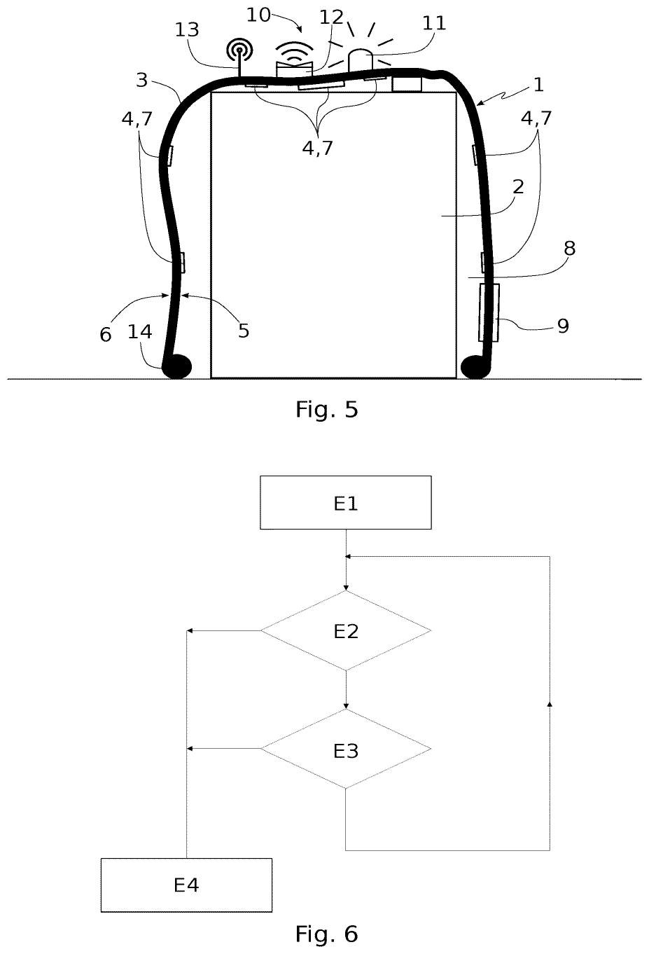

FIG. 5 is a schematic cross-sectional view of a battery 2 with a safety cover 1 in place. FIG. 5 illustrates the distribution of sensors 4, 7 around a portion of the battery 2.

The sensor(s) 4, 7 may be the only sensors present in the cover, or may be combined with additional sensors.

As illustrated in FIG. 3, the safety cover 1 may include at least one carbon oxide sensor 7 disposed on the inner face 5 of the flexible membrane 3. In one example, the safety cover 1 has a single carbon oxide sensor 7 that measures the evolution of the gas content from the carbon oxide family present in the air of the confined atmosphere 8.

The safety cover 1 may comprise as many carbon oxide sensors 7 as necessary, it being understood that a single carbon oxide sensor 7 is sufficient for many battery sizes, as gases diffuse rapidly throughout the volume of the confined atmosphere 8. In the case of a single carbon oxide sensor 7, the central position is preferred.

In a preferred embodiment, the carbon oxide sensor is a carbon dioxide and carbon monoxide sensor, which is therefore suitable for measuring the level of carbon dioxide and carbon monoxide in the air of the confined atmosphere 8.

The carbon oxide sensor(s) 7 may be made using any known technology that allows such detection, and may be attached to the flexible membrane 3 in any known manner, such as the temperature sensors 4, so that the active part of the sensor is located on the inner face 5 of the flexible membrane 3.

The carbon oxide sensor(s) may be the only sensors present in the cover, or may be combined with additional sensors. In the embodiment shown, however, they are combined with temperature sensors.

The safety cover 1 also includes a control module 9 which is connected to the various sensors 4, 7. The control module 9 may be a conventional electronic device, for example a microcontroller and its peripherals, for collecting and processing the signal from the sensors 4, 7.

The control module 9 may be able to detect an event relating to the temperature of the confined atmosphere 8 (and therefore of the battery), whether localized or diffuse, when the rate of increase of this temperature exceeds a predetermined threshold. The array of temperature sensors 4 thus makes it possible to grid the confined atmosphere 8 (which is confined by the flexible membrane 3) so that a hot spot at the battery 2 can be detected, thanks to the array of temperature sensors 4 which provides a mesh of the temperature evolution around the hot spot. The control module 9 is therefore able to collect instantaneous temperature information from the temperature sensors 4, to detect and determine the rate of temperature increase, and to compare it to the predetermined programmed threshold.

For example, this predetermined threshold may be set at 1° C. per second. Thus, the control module 9 detects a temperature event when it identifies a temperature increase of more than 1° C. per second on at least one of the temperature sensors 4.

Even when the safety cover is only equipped to measure temperature, using one or more temperature sensors 4, it can quickly detect and alert to abnormal heating and/or the start of a fire.

The control module 9 may also be able to detect an event relating to carbon oxide gas in the confined atmosphere 8, when the rate of increase in the level of carbon oxide gas exceeds a predetermined threshold. The control module 9 is therefore able to collect instantaneous level information from the carbon oxide gas sensor(s) 7, to detect and determine the rate of increase of this level, and to compare it to the predetermined programmed threshold.

For example, this predetermined threshold may be set at 25 ppm per second for the rate of increase of carbon dioxide, and at 5 ppm per second for the rate of increase of carbon monoxide. Thus, the control module 9 detects a carbon oxide gas event when it identifies an increase in the respective level of these gases greater than 25 ppm per second or 5 ppm per second.

Even when the safety cover is only equipped to measure the carbon oxide level, using one or more carbon oxide sensors 7 in the confined atmosphere 8, it can detect and alert when there is a change in this level. This is typically representative of an event in the confined atmosphere that may be related to venting (also known as degassing) of a battery cell 2. In the case of a large battery, or one integrated into a massive system, this detection linked to gas diffusion may occur before a significant rise in the temperature of the system is detected, which is thermalized by the quantity of material present.

The safety cover 1 also includes an alert interface 10 which is designed to trigger an alert signal when the control module 9 detects at least one of the events described.

The alert interface 10 is designed to alert a user or an external device when the control module 9 detects one of said events. The alert interface 10 may consist of any conventional electronic or microelectronic device and comprises at least one alert means. For example, the alert interface 10 may include a visual alert device 11, an audible alert device 12, and/or a remote alert device 13.

The visual alert device 11 may be, for example, one or more LEDs indicating the triggering of an alert. This visual alert device 11 may also provide more complete information via a display, a colour code, or a flashing code, to give details about the alert in question, as explained below.

The audible alert device 12 may be any known means capable of emitting an audible warning, alarm, etc. This audible alert device 12 may also provide more complete information by emitting an auditory code, or in natural language, to give details about the alert in question, as explained below.

The remote alert device 13 may be any known device suitable for remote communication, for example a radio communication device, on a suitable frequency (e.g. Wi-Fi, Bluetooth, 4G, etc.) or a wired device connected to remote alert means in relation to the safety cover, and allowing in particular connection to a computer network and the Internet, for information delivered to fixed or mobile terminals.

The alert interface 10 may also include any other suitable alert device for sending a specific alert to a user or system.

When the control module 9 detects one of the above events, the alert interface 10 is activated and emits an alert signal indicating that the battery 2 has been detected as presenting a danger:
localized thermal runaway of the battery (detected by a sharp increase in temperature on certain temperature sensors 4 only);
generalized thermal runaway of the battery (detected by a sharp increase in temperature across the entire array of temperature sensors 4);
a fire within the battery (detected by a very significant and very localized increase in temperature and a rapid increase in the level of carbon oxide gas).

The safety cover 1 may also include additional sensors, which can either add further alerts or provide information on the battery's history. In the examples described, the additional sensors may be:
light intensity sensors, for detecting electric arcs produced by the battery;
accelerometers or other vibration sensors for detecting shocks to the battery;
volatile organic compound (VOC) sensors, for detecting electrolyte leaks;
battery ambient temperature sensors, located on the outer face 6 of the flexible membrane 3, for detecting situations where the battery is exposed to an ambient temperature that could damage it.

The corresponding alerts can be issued by any means chosen from the alert interface options, with visual or audible signals, or information transmitted by radio communication.

The light intensity sensors are located on the inner face 5 of the flexible membrane 3 and connected to the control module 9 so that the control module 9 can detect an increase in light intensity beyond a predetermined threshold (e.g., a threshold of 200 lux per second). Such a light intensity sensor is able to detect an electric arc occurring at the battery 2.

Preferably, such light intensity sensors are also arranged in the form of an array of light intensity sensors, for example at a density of 1 sensor per 50 cm² of surface area of the flexible membrane 3.

Preferably, the flexible membrane 3 is also opaque so that the confined atmosphere 8 remains in darkness and is not disturbed by light external to the safety cover 1.

The accelerometers are arranged on the flexible membrane 3, as close as possible to the battery, and connected to the control module 9 so that the control module 9 can detect shocks of an intensity greater than a predetermined threshold and count these shock events (for example, a threshold of 8 g of acceleration). Such accelerometers are suitable for detecting shocks that cause damage to the battery, which may pose a hazard.

The measurement can be performed by a single accelerometer.

The ambient temperature sensor(s) are located on the outer face 6 of the flexible membrane 3 and are connected to the control module 9 so that the control module 9 can detect a temperature above a critical threshold. Lithium-ion batteries, for example, are sensitive to high temperatures. The alert threshold can be set at 50° C., for example.

The safety cover 1 also preferably includes means to ensure adequate coverage of the battery 2 and to ensure that the flexible membrane 3 is held in place. The safety cover 1 may advantageously include a weighted peripheral rim 14 for this purpose. For example, the flexible membrane 3 may have a hem around its perimeter, and this hem is filled with a flexible or sprayed ballast material, as shown in FIG. 3. This weighted peripheral rim 14 ensures that the battery 2 is covered, as illustrated in the cross-sectional view in FIG. 5.

FIG. 6 illustrates the method according to the invention, which allows the battery 2 to be monitored using the safety cover 1.

The battery 2 is first identified and a decision is made to monitor it. The battery is, for example, a battery with an unknown history (stock awaiting recycling), a battery involved in an accident, a battery that needs to be transported, etc.

In step E1 (FIG. 6), the safety cover 1 is placed over the battery 2 so that the flexible membrane 3 at least partially covers the battery 2.

In step E2, an assessment of the carbon oxide gas in the air of the confined atmosphere 8 is carried out. For example, the rate of increase in the CO2 and CO levels in this air can be measured and compared to its threshold value, which in this illustrative example can be 25 ppm per second and 5 ppm per second, respectively. If either of these two carbon oxide gases is detected with such a rate of increase, the control module 9 proceeds to step E4, in which the alert interface 10 is activated to express an alert relating to the start of a fire.

In step E3, an assessment is made of the temperature in the confined atmosphere 8 around the battery 2. For example, the rate of temperature increase around the battery 2 can be measured and compared to its threshold value, which in this illustrative example may be 1° C. per second. If such a rate of temperature increase is detected, the control module 9 proceeds to step E4, in which the alert interface 10 is activated to express an alert relating to one or more hot spots on the battery, or thermal runaway.

The control module 9 loops back to steps E2 and E3 until an E4 alert is given. The order of detection E2 and E3 is irrelevant. The temperature detection may be performed before the gas detection. The method may include only one of the steps E2 or E3.

The alert at step E4 can be given selectively, to provide more information about the event, giving details of the event detected thanks to the capabilities of the alert interface 10. The alert interface 10 is thus able to emit a signal representative of an alert indicating an alert level, i.e. providing additional information on the severity or type of alert.

The alert given by the alert interface 10 can thus present different, more precise information by emitting different light signals or sound signals for each type of alert, when the alert interface includes visual 11 or audible 12 alert devices, or by providing more information, for example on a screen or array, using the radio alert device 13 if present.

A visual alert using light signals combined with an audible alert, for example, is particularly effective in a storage area where many batteries are being monitored, as the user can immediately identify that an alert has been activated in the stock area (via the audible signal) and then identify which battery is affected (via the visual signal). Consulting a screen relating to the safety cover 1 of the battery concerned will ultimately provide a more accurate diagnosis of the event.

Similarly, the alert interface 10 can emit signals relating to the location of the hot spot on a large battery.

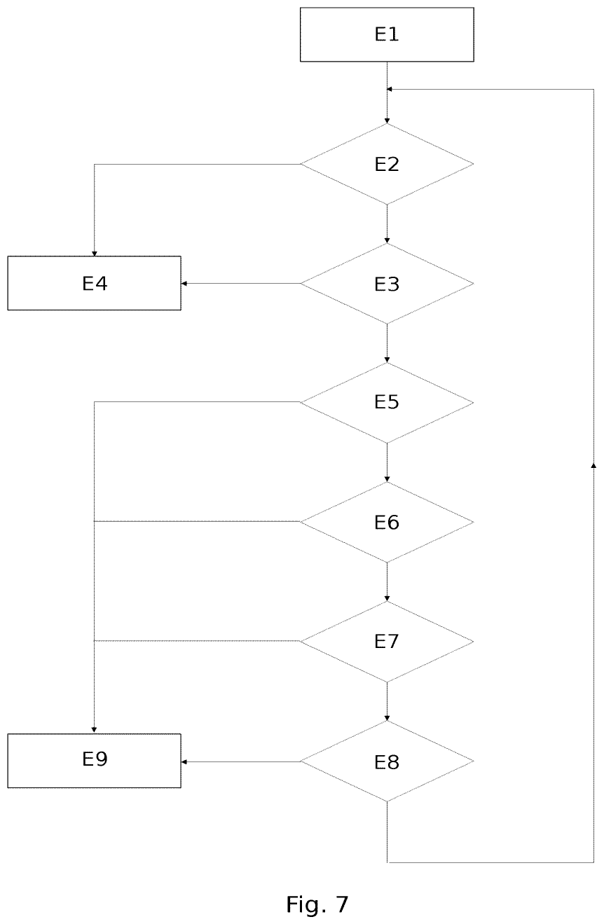

FIG. 7 illustrates another embodiment of the method, using the additional sensors described above. Thus, in addition to steps E1 to E4 described above, the method here comprises:
a step E5, in which the light intensity in the confined atmosphere 8 is assessed using the light intensity sensors. For example, the rate of increase in light intensity can be measured and compared to its threshold value, which in this illustrative example can be 200 lux per second. If such a rate of increase in light intensity is detected, the control module 9 proceeds to step E9, in which the alert interface 10 is activated to express an alert relating to an electric arc produced by the battery 2;
a step E6 in which an assessment of the shocks suffered by the battery 2 is carried out using accelerometers. For example, the intensity of the shocks suffered can be measured by the safety cover 1 and compared to its threshold value, which in this illustrative example may be 8 g. If such an impact is detected, the control module 9 proceeds to step E9 in which the alert interface 10 is activated to express a warning indicating that battery 2 has suffered an impact likely to damage it, and that it may therefore be faulty;
a step E7 in which an assessment of the presence of VOCs in the confined atmosphere 8 is carried out using the VOC sensors. For example, the presence of VOCs can be measured and compared to its threshold value, which, in this illustrative example, can be a floor value depending on the sensors that detect the mere presence of VOCs in the atmosphere 8. If such a presence of VOCs is detected, the control module 9 proceeds to step E9, in which the alert interface 10 is activated to express an alert relating to an electrolyte leak in the battery 2;
a step E8 in which an assessment of the temperature experienced by the battery 2 is performed using ambient temperature sensors. For example, the ambient temperature outside the confined atmosphere can be measured and compared to its threshold value, which in this illustrative example can be 50° C. If such a temperature is detected, the control module 9 proceeds to step E9, in which the alert interface 10 is activated to issue a warning indicating that the battery 2 has been subjected to an excessively high temperature that is likely to damage it.

In addition, the alert device 10 may also include a cut-off module able to disconnect battery charging equipment. This cut-off module is designed to be connected, for example, to a battery charger, or to the power supply of such a charger, for a battery 2 that is to be monitored during charging. This may, for example, be a plug-in socket with a controlled relay to be inserted between a wall socket and the battery charger's power cable. Thus, according to a variant of the method, in the event of events being detected, in addition to activating alerts, the alert interface 10 can cut off the charging current to stop or slow down the incident in progress. An external safety system, such as a fire extinguishing or inerting system for the site, can also be triggered via an interface on the alert device, for example via a dry contact relay that is activated during an alert.

Activation of the alert interface 10 can lead to the emission of a signal that not only represents an alert indicating the detection of one of said events, but also represents the location of the sensor zone in which said event was detected. The alert interface 10 is then able to emit a signal representative of the location of the event, for example to locate the battery concerned when the safety cover 1 covers several batteries, or to locate the area concerned on a battery. To implement this feature, the safety cover preferably comprises several sensors, or even an array of sensors.

Variant embodiments may be implemented. For example, the safety cover may comprise a flexible membrane made of any other suitable material, woven or non-woven (deformable polymer, etc.), with sufficient flexibility to cover a battery 2.

Similarly, any type of battery, sub-assembly comprising batteries, or even vehicles equipped with such batteries, needing to be monitored can benefit from the safety cover 1, which is then adapted to the appropriate dimensions.

The safety cover may also include any other known sensor that can add additional functions, connected to the control module 9 and the alert interface 10.

1. A safety cover for monitoring electrochemical batteries, wherein the batteries comprise:
a flexible membrane, able to at least partially surround at least one electrochemical battery to be monitored by defining a confined atmosphere, said flexible membrane having an inner face intended to face the electrochemical battery, and an outer face intended to face the outside;
at least one temperature sensor disposed on the inner face of the flexible membrane;
a control module connected to the temperature sensor and able to detect an event involving an increase in the temperature of the confined atmosphere at a rate exceeding a predetermined threshold;
an alert interface able to emit a signal representative of an alert when the control module detects said event.

2. The safety cover according to claim 1, wherein said at least one temperature sensor comprises an array of temperature sensors arranged on the inner face of the flexible membrane and connected to the control module.

3. The safety cover according to claim 2, wherein the array of temperature sensors includes a surface matrix of temperature sensors arranged on the surface of the inner face of the flexible membrane.

4. The safety cover according to claim 2, wherein the array of temperature sensors has a surface density of sensors of substantially 1 sensor per 10 cm² on at least a portion of the inner face of the flexible membrane.

5. The safety cover according to claim 1, wherein the alert interface is able to emit a signal representative of an alert indicating the location of the sensor area in which said event was detected.

6. The safety cover according to claim 1, wherein the safety cover further comprises at least one carbon oxide sensor arranged on the inner face of the flexible membrane, and wherein the control module is connected to said at least one carbon oxide sensor and is able to detect an event of an increase in the level of carbon oxide gas in the confined atmosphere at a rate greater than a predetermined threshold, the alert interface being able to emit a signal representative of an alert when the control module detects said event.

7. The safety cover according to claim 6, wherein the carbon oxide sensor is a carbon dioxide and carbon monoxide level sensor.

8. The safety cover according to claim 1, wherein the flexible membrane is formed of a flexible material of which the melting point is higher than a predetermined temperature. [symbol] claim 1, wherein the flexible membrane has a weighted peripheral rim.

10. The safety cover according to claim 1, wherein the alert interface comprises an audible alert device or a visual alert device.

11. The safety cover according to claim 1, wherein the alert interface comprises a remote alert device.

12. The safety cover according to claim 1, wherein the alert interface is able to emit a differentiated signal depending on the event detected by the control module.

13. The safety cover according to claim 1, wherein the flexible membrane is formed of a fabric of glass fibres, or silica, or silica aerogel.

14. The safety cover according to claim 1, wherein the flexible membrane is coated with a polymer or a mineral. [symbol] claim 1, wherein the safety cover further comprises an array of light intensity sensors arranged on the inner face of the flexible membrane and wherein the control module is able to further detect an event of an increase in light intensity at a speed greater than a predetermined threshold.

16. The safety cover according to claim 15, wherein the array of light intensity sensors has a surface density of sensors of substantially 1 sensor per 50 cm² on at least a portion of the inner face (5) of the flexible membrane.

17. The safety cover according to claim 1, wherein the flexible membrane is opaque.

18. The safety cover according to claim 1, wherein the safety cover further comprises at least one accelerometer disposed on the inner face of the flexible membrane, and wherein the control module is able to further detect an impact event subjected to an acceleration greater than a predetermined threshold.

19. The safety cover according to claim 1, wherein the safety cover further comprises at least one volatile organic compound sensor disposed on the inner face of the flexible membrane and wherein the control module is able to further detect an event involving the presence of a carbonate compound.

20. The safety cover according to claim 1, wherein the safety cover further comprises at least one ambient temperature sensor disposed on the outer face of the flexible membrane, in contact with the ambient air, and in that the control module is able to further detect an event of an increase in the ambient temperature beyond a predetermined threshold.

21. The safety cover according to claim 1, wherein the alert interface further comprises a cut-off module able to disconnect battery charging equipment and/or to trigger a battery inerting system, and/or to trigger a battery extinguishing system.

22. The safety cover according to claim 1, wherein the alert interface is able to emit a signal representative of an alert indicating an alert level.

23. A method for monitoring an electrochemical battery, with a safety cover according to claim 1, wherein the method comprises the following steps:
positioning the safety cover on the battery
detecting, using the control module, an event involving an increase in the temperature of the confined atmosphere at a rate greater than a predetermined threshold;
activating the alert interface.
